(19) United States
(12) Patent Application Publication
(10) Pub. No.: US 2023/0053870 A1
(43) Pub. Date: Feb. 23, 2023

(54) Microfluidic Device with Interface Pinning Vessels Within a Flow-Through Chamber, Kit for Forming, and Use of Same

(71) Applicant: National Research Council of Canada, Ottawa (CA)

(72) Inventors: Matthias GEISSLER, Boucherville (CA); Keith J. MORTON, St-Bruno-de-Montarville (CA); Teodor VERES, Montreal (CA)

(73) Assignee: National Research Council of Canada, Ottawa, ON (CA)

(21) Appl. No.: 17/796,025

(22) PCT Filed: Feb. 8, 2021
(86) PCT No.: PCT/IB2021/051007

§ 371 Date: Jul. 28, 2022

Related U.S. Application Data
(60) Provisional application No. 62/971,539, filed on Feb. 7, 2020.

(51) Int. Cl. B01L 3/00; G01N 33/543

(52) U.S. Cl. CPC B01L 3/502707; G01N 33/54386; B01L 2200/10; B01L 2300/0864; B01L 2400/0409; B01L 2400/0406; B01L 2300/0816

(57) ABSTRACT

A technique for detection of probes in a microfluidic flow-through chamber involves a plurality of interface pinning reaction vessel formed by micro- or nano-structured relief patterning of a substrate. The relief patterning increases a surface area locally, and defines a plurality of separated interface pinning reaction vessels. The marked detection protocol may be supplied on a single layer of a stacked microfluidic chip, or the chamber may constitute a whole layer. The chip may be designed to be driven mechanically, pneumatically, hydraulically, centrifugally or by capillary action. Each vessel allows for a high density of probes, an effective region for developer-type or fluorescence-based marking, and efficient readout. Suitable probe liquids can be self-limiting to fill one vessel. Suitable developer liquids avoid dye bleeding across vessels during washing.

### FIELD OF THE INVENTION

The present invention relates in general to microfluidic devices for multiplex assays, and in particular to a microfluidic chip with multiple interface pinning reaction vessels for concentrating and arraying bound targets/analytes in respective regions of a flow-through chamber.

### BACKGROUND OF THE INVENTION

Multiplex assays are important for a wide variety of testing and studies in biological sciences, pharmacology, food and water testing, and clinically. Particularly useful are systems that allow for a test sample to be tested for presence of a few to many species (e.g., up to 384 for standard well plates) with the test sample provided to a plurality of target regions in sequence as in a flow through chamber, as opposed to dividing the test sample and sending each fraction to a different target region. Flow-through processing allows for a more parsimonious use of the test sample, and is particularly required when low volumes of test samples are available.

Historically, multiplex assays have mostly been done using well plates and have required intensive human intervention, or expensive robotic manipulators. The use of standard well plates offers limited potential for miniaturization, as a volume and spacing of the well plate needed for read-out and pipetting (while avoiding cross-contamination risks) limit the well plates to a certain size and volume of liquid.

A critical step in multiplex assays is read-out. Once targets are bound to capture moieties in respective wells, it is desired to inspect the regions to determine the presence of the target from the sample. This is why it is important for wells to concentrate and spatially array the bound targets/analytes. A density of bound targets when inspected from a predefined vantage is essential for easy, reliable, low-cost, inspection. For example, the bound targets may be fluorescently labelled or dyed for visual read out, or other read out technologies could be used.

Colorimetric assays are widely used as diagnostic tools for detecting target analyte through the formation of a colored reaction product. They are commonly performed using standard (e.g., 96 well) well plates (e.g., made of polystyrene) where capture probes are immobilized at the bottom of each well. Solutions are pipetted in and out of the wells to expose the probes to i) a sample, possibly or certainly containing analyte, ii) conjugated detection antibody (e.g., conjugated with HRP), and iii) developing agent (e.g., TMB) as well as iv) rinse buffer to perform wash steps in between. Although the colorimetric reaction happens at the interface (where probe-analyte conjugate is located), the forming product typically dissipates in solution. Colorimetric assays generally benefit from automation for high-volume test runs. Read-out is accomplished through imaging (photographic analysis, absorbance spectra) for each well.

Test strips can be used alternatively to well plates. These can reduce a volume of the sample liquid, and still provide good read-out capabilities with well selected visualization reagents. These typically use capillary effect to draw sample liquid from an input area into contact with one or more probe areas. However, there are challenges with manipulating small volumes of liquids, and avoiding cross-contamination. It can be difficult to supply a same small volume to different regions for multiplex assay when capillarity is the sole driver of the liquid, and reliable operation generally calls for an oversupply of liquids to ensure that the liquid covers the test strip. Test strips often need handling procedures to avoid contact with sensitive regions prior to use. A range of binding assays are limited as many-step processes (e.g. sample supply, wash, antibody conjugate delivery, wash, developer delivery and wash) are substantially precluded, even though these many-step processes may be more reliable. Finally, if sample prep is required, complex equipment may be called for that largely vitiate the portability, and efficiency advantages of test strips.

Paper, for example, has become another popular and widely used support for colorimetric tests (A. W. Martinez, S. T. Phillips, M. J. Butte, G. M. Whitesides “Patterned paper as a platform for inexpensive, low-volume, portable bioassays” Angew. Chem. Int. Ed. 2007, 46, 1318, Morbioli et al. “Technical aspects and challenges of colorimetric detection with microfluidic paper-based analytical devices (pPADs) — a review” Anal. Chim. Acta 2017, 970, 1, Gong & Sinton “Turning the page: advancing paper-based microfluidics for broad diagnostic application” Chem. Rev. 2017, 117, 8447). Paper can be implemented either in the form of a continuous test strip that contains multiple probes immobilized with spatial control, or as isolated segments each of which having been modified with a respective probe. It is possible to structure paper using wax or resin in order to provide guidance and directionality to flowing liquid (Martinez et al. 2007).

There are many patents on test strips (e.g. U.S. Pat. Nos. 4,361,537, 4,960,691, 4,168,146, CA 2493616, CA 2375034), including CA 2637974 and CA 2272260 which teach multiple analyte binding assays. Test strips typically offer open structures for conducting the sample liquid between various reaction and (sometimes distinct) read-out zones, under the influence of capillary action. WO03/012443 to Chan teaches a paper-based membrane (test strip) for a rapid diagnostic device in which a liquid test sample is assayed to detect a target analyte. Chan teaches that porosity of the membrane has a large influence on flow rate through the membrane, and sensitivity of the assay. The larger the pore size, 1—the faster the flow rate (and the shorter the interaction time), and 2—the less the surface area of the receptor molecules. Both of these tend to decrease sensitivity ceteris paribus.

The use of plastic based, microfluidic systems for multiplex assay holds great potential for developing point-of-care technology. However, incorporation of these detection schemes into microfluidic devices remains challenging, mainly because current microfluidic systems lack the capacity (reservoir depth) and target density at the same time, both of which are required in order to supply reagents for development and to ensure accumulation of colored reaction product for providing strong, measurable signals. Microfluidic chips are generally produced by relief patterning of a surface of a film to define a network of chambers and channels, and then covering this surface with a lid to enclose the chambers and channels. This does not usually permit deep reservoirs as are used routinely in well plates. Furthermore, flow of solution (as it occurs in microfluidic systems) generally leads to dissipation of reaction product and is therefore detrimental to detection, which is not a problem if separate well plates are used. It is a challenge for microfluidic devices to provide readable regions with the depth limitations, and to provide flow-through chambers while, at the same time, retaining colorimetric products within the regions.

One prevailing strategy to circumvent the problems of dissipation and limited depth, is the use of porous materials that can act as a three-dimensional reaction matrix. For example, an assay has been demonstrated as a Health Canada-approved method for the detection of enterohemorrhagic E. coli (EHEC) colony isolates on a cloth substrate (B. Blais et al. In: Compendium of Analytical Methods 2013, Vol. 3. Laboratory Procedures of Microbiological Analysis of Foods, MFLP-22). Key marker genes for this organism (eae, rtbO157, vt1, vt2 etc.), amplified in a multiplex PCR process, incorporate a detectable digoxigenin (DIG) label, which is revealed in an immunoenzymatic process using TMB conversion after hybridization with target-specific oligonucleotide capture probes (A. Martinez-Perez and B. W. Blais “Cloth-based hybridization array system for the identification of Escherichia coli O157:H7” Food Control 2010, 21, 1354). Applicant has demonstrated a colorimetric detection assay for pathogenic E. coli by integrating a polyester cloth substrate on a microfluidic chip (M. Geissler, L. Clime, X. D. Hoa, K. J. Morton, H. Hebert, L. Poncelet, M. Mounier, M. Deschênes, M. E. Gauthier, G. Huszczynski, N. Corneau, B. W. Blais, T. Veres “Microfluidic integration of a cloth-based hybridization array system (CHAS) for rapid, colorimetric detection of enterohemorrhagic Escherichia coli (EHEC) using an articulated, centrifugal platform” Anal. Chem. 2015, 87, 10565).

The integration of paper or cloth (fabric) in a microfluidic chip complicates chip design, fabrication and assembly, and therefore adds to the cost per assay. Surface modification of cloth or paper to localize capture probes requires processing that is not compatible with low-cost fabrication schemes unless a very large production run is envisaged. Integration of separate, small swatches of fabric into a microfluidic chip with a required alignment precision, is generally time-consuming and expensive. Bonding and functionalization of the swatches also add substantial costs to the process.

Another approach is to define a 3D scaffold with a gel-type structure. For example, US 2011/0186165 to Borenstein (165) uses hot embossing of thermoplastic films to form microfluidic channels and chambers, and then injects a gel matrix, in at least one chamber between two channels. Unfortunately, supplying and affixing gels, can be a laborious and capricious exercise (see [0049]-[0050]). The gel may have porosity, permeability or surface wetting properties that change with time, and are liable to a host of stability issues that may make their non-immediate use unreliable. As gels are essentially random arrays of very fine structural members, they have very high surface area for interacting with a liquid, which is good for assay efficiency, but are very difficult to wash between steps, and they are prone to clogging or blocking. Furthermore gels may not have a desired reference surface contrast, and may absorb or block light from some reporter molecules, thereby limiting detection alternatives.

'165 refers to microfluidic devices with “non-uniformly treated and/or patterned interior surfaces”. Surface treatment and/or patterning is said to include chemical and/or topographical surface modifications; the chemical modification to include treatments and/or coatings with inorganic or organic (e.g. antibodies or proteins) substances. The interior surface of a microfluidic device includes the walls of the microchannels and walls of the gel-holding chamber. Note these features on interior surfaces are not themselves said to alter fluid dynamics, and once filled with gel, the porosity of the gel would substantially determine fluid conductance. Patterning implies repetitive (though not necessarily perfectly regular) surface modifications. For example, in some embodiments, one or more microchannel walls feature chemically (including, e.g., biologically) treated islands, or non-treated islands on an otherwise treated wall. Certain interior surfaces may be topographically structured, e.g., with microposts. According to '165, microposts disposed at the top and bottom surfaces of a gel-containing chamber may serve to hold the gel in place or to support cells, or stiffen walls to improve cell adhesion.

Apart from '165, which teaches the use of posts for cell support, and to confine gel, there is a great deal of knowledge in the art surrounding arrays of posts or like features in microfluidic chips. While U.S. Pat. No. 6,210,986 to Arnold et al. (986) seems to be committed to using ceramic and glass substrates, '986 teaches etched microfluidic structures with an array of posts within a microfluidic channel used as flow guides, material supports, or as the porous phase for chromatographic separation. Pillar arrays are also known for: 1—force measurements (J. C. Doll et al. “SU-8 force sensing pillar arrays for biological measurements” Lab Chip 2009, 9, 1449) and cell mechanics studies (S. Ghassemi et al. “Fabrication of elastomer pillar arrays with modulated stiffness for cellular force measurements” J. Vac. Sci. Technol. B 2008, 26, 2549); 2—surface-enhanced Raman scattering—if the pillars are metallized and nano-scale (e.g. Applicant's WO 2012/122628entitled “Microfluidic system having monolithing plasmonic nanostructures”; Y. Q. Wang et al. “Size-dependent SERS detection of R6G by silver nanoparticles immersion-plated on silicon nanoporous pillar array” Appl. Surf. Sci. 2012, 258, 5881; and J. C. Caldwell et al. “Plasmonic nanopillar arrays for large-area, high-enhancement surface-enhanced Raman scattering sensors” ACS Nano 2011, 5, 4046); 3—particle separation based on Deterministic Lateral Displacement, if the pillars have a prescribed layout and dimensions relative to the fluid and particles (e.g. L. R. Huang et al. “Continuous particle separation through deterministic lateral displacement” Science 2004, 304, 987; D. W. Inglis et al. “Critical particle size for fractionation by deterministic lateral displacement” Lab Chip 2006, 6, 655; and J. McGrath et al. “Deterministic lateral displacement for particle separation: a review” Lab Chip 2014, 14, 4139); 4—immunomagnetic capture, if the pillars are coated and magnetized (e.g. L. Malic et al. PCT/1B2019/056616, and L. Malic et al. “Polymer-based microfluidic chip for rapid and efficient immunomagnetic capture and release of Listeria monocytogenes” Lab Chip 2015, 15, 3994); 5—creating passive pumping elements/wicking to displace liquid in a microfluidic channel (e.g. M. Zimmermann et al. “Capillary pumps for autonomous capillary systems” Lab Chip 2007, 7, 119, and L. Gervais and E. Delamarche “Toward one-step point-of-care immunodiagnostics using capillary-driven microfluidics and PDMS substrates” Lab Chip 2009, 9, 3330); and 6—controlling interface adhesion in microfluidic blister seals (e.g. M. Janta et al. “Patterned film for forming fluid-filled blister, microfluidic blister, and kit and method of forming” US 2017/0291747) and valves (J.-C. Galas et al. “Semipermanently closed microfluidic valve” U.S. Pat. No. 9,435,490).

While it might appear obvious to place a test strip into a suitably sized cavity of a microfluidic chip to provide all the advantages of the test strip in terms of liquid control and read-out, and freedom from handling restrictions (once installed): 1—test strips are only designed for a narrow range of test protocols, and microfluidics can permit a broader range that includes some protocols that are better suited to some tests; 2—the costs of test strips are simply added to the costs of the microfluidic chip (with costs for assembly); 3—read-out of the test strip through the microfluidic chip might be more difficult; and 4—bonding and sealing of chips may be made more difficult by the inclusions as it is for fabric swatches.

Accordingly there is a need for a microfluidic chip adapted for multiplex assays that provides flow control and high density of targets without relying on exogenous scaffolds or materials inserted into the chip.

### SUMMARY OF THE INVENTION

Applicant has devised a flow-through chip that: can be easily functionalized to provide a plurality of probes in respective regions with low risk of cross-contamination; has high surface area for the probes in each region, for improved assay efficiency and read-out of minuscule volumes; is integrated with a microfluidic chip; and allows for long shelf-life usage. The microfluidic chip structure allows for enclosed testing spaces that have reduced handling limitations, and allows for a wide range of assay processes, including colorimetric or developer-based assays. The microfluidic chip structure enables lower volume assays with good readout because of a high surface area within interface-pinning reaction vessels that allow for high target density.

Accordingly a kit for forming a microfluidic chip is provided, the kit comprising a substrate having a surface with topographical relief bearing at least 4 relief patterned regions, each defining a respective interface-pinning reaction vessel covering a footprint area of less than 15 cm²; and a part with a covering surface dimensioned for sealing against the substrate to cover the substrate to enclose at least a single flow-through chamber that includes the vessels.

Each region may preferably extend 0.1 to 50 mm in both planar directions, and may have a surface area that is at least 1.2 times, and more preferably 1.6 times, or 2-50 times its footprint area. Each region may be separated from each neighbouring region by segments of the surface that have a ratio of surface area to footprint that is no more than 1.1. Each segment may separate the neighbouring regions by a distance that is greater than: 0.1 mm; or 5% of a mean of the extents of the neighbouring regions in the planar directions. As such each region is an interface-pinning reaction vessel for many fluids, separated from other regions to both avoid cross-contamination, and to facilitate readout.

The chamber may have at least one ingress from a microfluidic network of the chip formed by the kit, the microfluidic network comprising at least two microfluidic channels coupling two different reservoirs with the ingress. The microfluidic network may comprise two subnetworks: a marking network equipped for performing a marking process within the chamber; and a prep network equipped for treating a test sample.

The part may be a first film, and the covering surface, a side of the first film. The side of the first film, or the substrate surface, may be relief patterned to define one or more of: one side of the chamber, the relief-patterned regions, one side of the ingress, the whole ingress defined as a through-bore of the first film, and at least part of the microfluidic network. The relief pattern may define at least one microfluidic blister for retaining a liquid.

The substrate may be in the form of a second film; the kit further comprises a third film; and at least one of the first, second or third films, has at least one through-bore via for coupling two microfluidic networks when the films are stacked and bonded.

At least one of the substrate and the part is preferably transparent to inspection at a wavelength, and a chip produced by sealing the surface and the covering surface (and possibly other steps) permits inspection of the vessels through the transparent material. Preferably the transparent material is sealed to a material that is reflective or opaque to the inspection wavelength, to improve imaging of the vessels.

The kit may further comprise supplies of at least 3 probes. The probes may be supplied by functionalizing each of the vessels with a respective one and only one of the at least 3 probes.

The substrate may be composed of a cyclic olefin copolymer-, polystyrene-, or polylactic acid-based polymer and the functionalization may be consistent with formation by oxygen plasma surface activation or UV/ozone surface activation. Prompt reaction with cyanogen bromide, or silanes (aldehyde, epoxy, or amine in conjunction with gluteraldehyde) and binding of the probe.

The probe may be supplied (for example prior to assembly of the chip), carried by a liquid in a fluid-tight container, the liquid having a contact angle and viscosity allowing for spontaneous spreading of the liquid across the region, and a volume sufficient to cover the region, but insufficient volume to overcome interface pinning, whereby the liquid, if it meets any part of the region, is self-limited to substantially covering that region.

The kit may further comprise at least one marking liquid, such as one or more of: a developer; a conjugated detection antibody with a target-specific binding moiety; a wash buffer; a hybridization solution; formaldehyde; and a PCR product contained within a microfluidic chamber of a chip formed with at least the substrate and the cover. If the substrate is a cyclic olefin copolymer, the developer may be 3,3′,5,5′-tetramethylbenzidine.

The kit may be assembled to form the chip. The chip may be loaded with a sample and/or other fluids for the assaying, and each vessel is preferably functionalized. The chip may be a centrifugal microfluidic chip, designed for mounting at a particular axis-relative position and operated to drive fluids through the prep and marking networks, or may have manual, or pressurized supply couplings for driving fluids.

Also accordingly, a method for assaying on a microfluidic chip is provided, the method comprising: providing a microfluidic chip, the chip composed of a cyclic olefin copolymer, and having at least one flow-through chamber defined on a single surface thereof in topographical relief, the chamber bearing at least four relief patterned regions, each defining a respective interface-pinning reaction vessel functionalized with a respective probe; supplying a test sample into the flow-through chamber, so that the test sample flows over each of the regions; supplying rinse buffer to wash unbound analyte off the surface; supplying a detection antibody conjugated with an enzyme; supplying rinse buffer to wash excess detection antibody off the surface; and supplying a developer to all vessels by flowing a developer agent through the chamber.

The method may further comprise functionalizing the respective regions, prior to forming the chip, by enclosing a relief-patterned substrate by dispensing a droplet anywhere within the region, allowing the droplet to spread across the region as a liquid, adhering a chemical species in the liquid over the surface of the interface pinning reaction vessel, and removing excess liquid or residue, for example by: evaporating a solvent or carrier of the liquid; heating the region to above a boiling point of the solvent or carrier (for example 60° C. for 1-10 min); or rinsing with buffer with a surfactant, and drying. The rinsing with buffer may comprise: dispensing a droplet of the buffer with surfactant into each region respectively, allowing the buffer to dissolve or suspend any unbound probe or reaction product, and wicking the buffer out of each of the regions without mixing the respective droplets; or flooding the regions with the buffer and surfactant, allowing the dissolution or suspension of any unbound probe or reaction product, and extracting the buffer from the regions.

The developer agent may produce a dye that has low solubility in a cleaning solution, while the developer agent itself is highly soluble in the cleaning solution, and, if so, the method may further comprise flowing the cleaning solution through the chamber after supplying the developer.

The developer may comprises TMB; the rinse buffer, PBST; a hybridization solution may contain formaldehyde; and the conjugated detection antibody may have a target-specific antibody moiety and a conjugated HRP enzyme.

Further features of the invention will be described or will become apparent in the course of the following detailed description.

### BRIEF DESCRIPTION OF THE DRAWINGS

In order that the invention may be more clearly understood, embodiments thereof will now be described in detail by way of example, with reference to the accompanying drawings, in which:

FIG. 1 is a schematic illustration of substrate relief patterning to form a blister and finger-pump actuated 6-vessel microfluidic chip in accordance with a first embodiment of the present invention;

FIG. 2 is a schematic illustration of substrate relief patterning to form an externally pumped 6-vessel microfluidic chip layer in accordance with a second embodiment of the present invention;

FIG. 3 is a schematic illustration of substrate relief patterning to form a centrifugal microfluidic chip with 7 pressure controlled ports and 8 vessels, in accordance with a third embodiment of the present invention;

FIG. 4 is a schematic illustration of substrate relief patterning to form a centrifugal microfluidic chip designed for an articulated centrifugal blade bearing 8 vessels, and providing a low complexity mixing processing area;

FIG. 5 is a schematic illustration of substrate relief patterning to form a microfluidic chip for articulated centrifugal operation, the chip having a marking area, and providing a via coupling to another layer of a microfluidic chip for receiving liquid subjected to centrifugal microfluidic processing;

FIG. 6 is a schematic illustration of substrate relief patterning to form a generic centrifugal microfluidic chip layer featuring 24 vessels, a via coupling to another layer of a microfluidic chip, and a waste reservoir;

FIG. 7 is a schematic illustration of a surface activation scheme used for the covalent attachment of amino-modified oligonucleotide probes on a cylic olefin-based substrate;

FIGS. 8,8A,8B,9 are images of a first example of the present invention, respectively showing: the chip as a whole; first; and second enlargements of a vessel surface patterning; and differentiated colorimetric readout;

FIGS. 10,10A-H,11A,B are images of a second example of the present invention used to evaluate feature density and colorimetric contrast, respectively showing: schematically, dimensions of lattice parameters varied in respective vessel patterns; eight micrograph images showing enlargements of respective micropatterns applied in respective vessels; a chip with two copies of the vessels; and the chip after colorimetric marking;

FIGS. 12A-D are images of a third example of the present invention showing a centrifugal-pneumatic chip for microfluidic processing, bearing an array of 7 vessels, centrally located on the chip, respectively showing: the whole chip; an enlargement of 3 of the vessels with a differentiated colorimetric marking; schematic fabrication detail, and a schematic layout of chambers, and interconnecting microfluidic channels;

FIG. 13 is a photograph showing differentiated colorimetric readout of a chip formed of polylactic acid;

FIG. 14 is a photograph showing differentiated colorimetric readout of a chip formed of polystyrene; and

FIGS. 15A-E is a panel of photographs showing fluorescent readout of respective vessels subject respectively to 1, 5, 10, 50 and 100 μg/ml concentrations of a capture probe.

### DESCRIPTION OF PREFERRED EMBODIMENTS

Herein a kit for forming a microfluidic chip, the kit as assembled to form a chip, and a method of using the chip, are provided. The chip may be operated by centrifugal, pneumatic, mechanical, electroosmotic, electrostatic/electrowetting, or capillary forces, and may be operated by any combination of such forces, but has a flow-through chamber with capillary force engineered detection area called an interface pinning reaction vessel in a flow through-chamber. A protocol for colorimetric assay is also provided for a chip composed of a cyclic olefin copolymer (COC) such as Zeonor™.

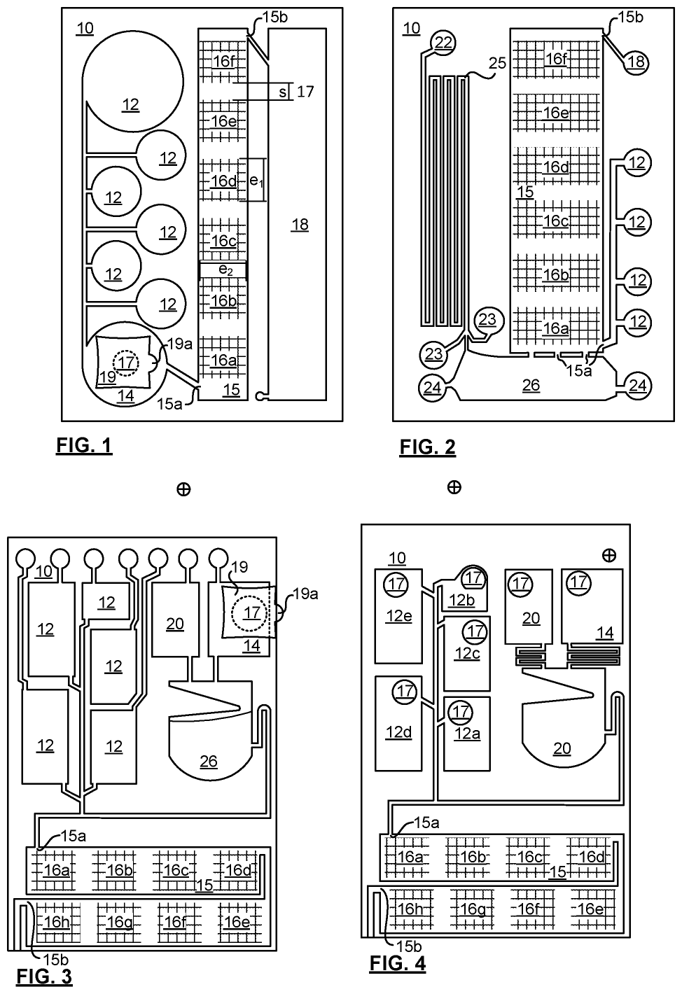

FIGS. 1-6 show a variety of patterned films for forming microfluidic chips. In these variants, common reference numerals identify equivalent or substantially equivalent features, and their descriptions will not generally be repeated herein. Each variant has one or more independent features that are intended to be transposable on any of the other variants to produce further embodiments of the invention.

As is conventional, a stack of patterned films with suitable through-holes (vias) interconnecting channels of respective patterned surfaces can be assembled to produce a chip having a variety of functions. A simplest stack is a single patterned film with a cover. The layers may advantageously be composed of biocompatible plastics, and at least alternating layers may advantageously be thermoplastic elastomers as these can be patterned at a low cost, and form sealing bonds with many other materials, as taught in U.S. Pat. No. 10,369,566. In particular, Applicant has found that inclusion of two or more layers of hard thermoplastic layers, such as Zeonor, and at least one TPE layer between the each pair of Zeonor layers, such as an oil-free Mediprene™ (U.S. Pat. No. 9,238,346), is particularly efficient as: low cost patterning and bonding can be provided with excellent seals; a stiffness of Zeonor layers assists in registration and alignment of the chip or stack; and Zeonor can offer excellent transparency, while Mediprene can provide an opaque back drop for contrast.

The chip typically has a plurality of reservoirs (usually vented, or controllably vented see U.S. Pat. No. 10,702,868, FIG. 11) for holding reagents, and other fluids, and some mixing chambers, the chambers and reservoirs being interconnected by a network of channels. Some operating mechanisms for controlling fluid movements within the chip are reliant on embedded features such as conductive electrodes and leads therefor, valves, and surface treatments and/or patterning of surfaces (capillary force engineering) or walls of microfluidic channels or chambers. Others require a layout of the chip to have a consistent positioning of ports and channels with respect to chambers, so that a centrifugal field gradient can control movement. In general, closed fluid dynamics require venting and a motive force. Furthermore, by incorporation of other elements into the chip, either between the layers of the chip, or around the chip, or by placing the chip in proximity to other elements, select regions of the chip, or content therein, may be acted upon (heated, cooled, irradiated, sonicated, activated, probed or sensed). Keeping such elements outside of the chip can permit cost-effective single-use chips, but for some applications local metallization, magnetization, treatment, or alteration, is needed to embed essentially passive elements within the chip to act upon the region or the content.

FIG. 1 is a schematic top plan view of a patterned film 10 showing all of the relief structure necessary to define a stand-alone microfluidic chip, in conjunction with a covering part or layer which is removed for illustration purposes. While, in principle, most microfluidic devices can function without an enclosure, the risks of contamination, evaporation issues, and reliability require covers for each patterned layer. Film 10 requires a cover (not shown) so blisters 12, and a pump region 14 can be pressed.

In accordance with the present invention, one of the cover and film 10, is transparent for inspection for read-out purposes, at least where required. Transparent for inspection may be transparent in one or more wavelength bands of an infrared, optical, or ultraviolet spectrum. While the whole cover or film 10 may be transparent, only an area covering interface pinning reaction vessels 16 need be transparent. Preferably only one of the substrate and cover is transparent to avoid issues with multiple images at different depths of view. So, the film 10 can be transparent and the cover can be reflective, or opaque, with a color and texture provided for good imaging contrast.

Film 10 as illustrated is adapted for mechanical actuation via an array of blister chambers 12, and a finger pump 14 on a supply side of the chip 10 (left as shown). The blister chambers may be small enough to be microfluidic chambers, though typically they have footprint of 0.1-20 cm², and volumes of 45 cm³ or more, which is large fora microfluidic chamber, but is nonetheless a reservoir for present purposes. A useful blister for microfluidic chips is disclosed in Applicant's U.S. Pat. No. 10,046,893, which is incorporated herein by reference. Relief patterning of vessels may procedurally or structurally resemble that of a gating region disclosed in U.S. Pat. No. 10,046,893.

Film 10 has a large flow-through chamber 15 provided in communication with the pump 14 via a microfluidic channel. Specifically, inlet 15a of the chamber 15 is part of a microfluidic channel communicating with the pump 14 on the supply side of the chip, and outlet 15b communicates with a chip waste port 18. Herein a microfluidic channel is understood to have a channel direction (at least locally), and a nominal channel width and depth perpendicular to the channel direction that are each less than 900 μm, more preferably from 1-300 μm. In the embodiment of FIG. 1, the nominal channel widths and depths may be from 50-500 μm.

At least one wall of the chamber 15 (floor as shown), defines 6 regions with respective micro- or nano-structured relief patterns respectively defining vessels 16. The relief structures, which may be recesses in, or protrusions from, the floor (or other wall) may have any form or arrangement, pillars, walls, fences, or lattices of any cross-sectional shapes or variances. The regions are preferably composed of a material that is naturally hydrophilic, or is coated or activated to induce capillary effect, at least with respect to liquids used for functionalization of the vessels 16. There are no transverse walls or partitions between the vessels 16 within the chamber 15 apart from micro- or nano-structuration which does not impede, but rather encourages, flow through the chamber 15. As such, the regions are separated by segments of the floor that are relatively smooth, either lacking in any patterned relief, or having much lower surface area for footprint area (e.g., having a relief pattern depth markedly less than that of the floor within one of the regions, or having fewer or otherwise lower surface area features).

Herein, a footprint area refers to a 2D area enclosed by a perimeter of the region. In FIG. 1, each region has a same perimeter, and the footprint area is its length (extent e₁) times its width (extent e₂). If the chip is 10 cm long and 6.8 cm wide, e₂ is about 1.2 cm, ei is about 1 cm, and the vessel footprints are 1.2 cm². However, the surface area of the vessel is at least 1.44 cm², more preferably at least 1.92 cm², and can be 1 to 2 orders of magnitude higher. For example, a regular array of pillars with uniform cross-section (square, regular polygonal, circular, etc.) of 40 μm perimeter, in a 2D packing (e.g. square, hexagonal, regular basis) with 50% duty ratio, and an aspect ratio of 8:1, has a surface area of 33× footprint. The region can be a regular tiling or an irregular mosaic. The pillars may be tapered or have varying cross-sections, or can be replaced by wall segments or branched structures. Every shape in positive relief has an analogous, equally useful, hole in negative relief.

The regions are shown arrayed in series, labelled a-f. While this may be convenient, in other embodiments the vessels 16 may be arranged in a rectangular array, a staggered or off-set array, or other arrangement that assists in readout. Preferably, a spacing (s) between the vessels 16 is regular. A spacing of at least 5% of the mean dimensions of the regions, and at least 0.1 mm may allow visualization. Better visual separation of the vessels may be provided with larger spacing, such as 0.2 mm and 15%, or 0.3 mm and 20%. As shown in FIG. 1, the separation is about 0.4 mm, which is about 40% of e₁ (which is the direction of the separation).

While the shapes, sizes, orientations and positions of the vessels 16 may be regular, within each region, there may be a variation in density of micropillars, microholes, or other microstructures. Specifically, it has been noticed that with uniform density of micropillars, in use, a peripheral area of such regions tend to be more strongly colored than an interior region, particularly if a probe density is weak. To improve consistency and ease of qualitative/quantitative assessment, within each region, various density gradients of features may be preferred to provide different wicking forces across each region. Furthermore, wicking forces can be directed by selective orientations of groups of features, to encourage flow across the chamber 15.

Furthermore, while the pillars may extend a full etch depth of the chamber 15, in some embodiments they advantageously only extend above or below a nominal floor of the chamber, by a fraction of the etch depth. For example, the micropillars, may extend between 20% to 100% of an etch depth. If microholes are used, they may extend 10%-200% of an etch depth of the floor from which they extend.

The chamber 15 preferably covers at least about 10% of the footprint of the chip, and preferably extends at least 60% of a length of the chip. As shown in FIG. 1 about 15% of the footprint of the chip is occupied by the chamber 15, and the chamber 15 extends about 90% of the chip's length. The six separated regions of micro- or nano-structured topography (i.e. the vessels) are arrayed in chamber 15, which is otherwise undivided in that no partitions or walls separate the vessels 16. The chamber 15 provides spacing 17 between the vessels 16 that facilitate read-out of colored or marked liquid retained therein. The chamber 15 may have an elongated shape with a large number of vessels 16 in a regular array. Each region is sized and separated to ensure inspection of the region independently of others, and are preferably maximally distributed within the chamber 12.

Any manner of marking of the film 10, or cover, may be used to facilitate identification of vessels 16 by their binding targets, for example. In particular, a cover may have a set of demarcating lines imprinted thereon for delineating respective vessels, to assist in viewing. If the cover is thin and transparent for inspection, reliable demarcation is possible with suitable alignment of the cover, over a range of viewing angles.

The embodiment of FIG. 1 can be sold as a patterned film 10, and the vessels thereof may be functionalized by a first buyer. The functionalized patterned film may be protected by applying the cover, temporarily or permanently, to form a chip. The covered film may be loaded so that blisters 12 retain various liquids (before or after application of the cover) by the first buyer, or a buyer of the functionalized patterned film. In general the liquids may be classed as one set of liquids necessary for sample preparation, and another set of liquids implicated in a marking process. In the illustrated embodiment, there may be no sample preparation on the chip. At this juncture, the chip may be ready for deployment. If the liquids are stable and the blisters are hermetically sealed, this chip may be stored for extended periods before use.

In use, a user may inject a sample into pump region 14, by peeling a resealable covering 19 (from a gripping ear 19a ), and then reseal it. Instead the covering may be designed to reseal from a puncture and the sample may be injected by a syringe. The resealable covering 19 preferably overlies a cover that defines a finger pump-area, although the covering is not in view. Preferably, the pump region 14 is filled above a minimum fill line, representing a minimum fill volume required for reliable testing, without overfilling. The user then presses on the pump region 14, and liquid, following the path of least resistance, passes through the ingress 15a, into chamber 15. By releasing the pump region 14 air may be aspirated into the pump region 14 to equilibrate pressure within the pump region 14 to minimize backflow into pump region 14. Alternatively, by bursting one of the blisters 12, and releasing pressure on the pump region 14, liquid from the blister can load into the pump region 14. Thus air aspiration may occur only at the blister, on the microfluidic channel by a user controlled valve, or at the pump region 14, and as a result each delivery of liquid may be separated by an air plug, or may be incorporated into a train.

A volume of the blisters 12 may be an integer number of a volume within chamber 15, whereby content of each blister (or an equal division thereof) may, in turn, fill the chamber 15 in accordance with a prescribed protocol, although this is not necessary, and one vessels may be exposed to one agent while another is exposed to another.

Herein a number of variants of the film 10 are provided. While these may share little in form, they all have in common a flow-through chamber 15 with four or more vessels 16 defined by higher surface area relief patterns, and some kind of microfluidic network that couples two or more reservoirs to an ingress 15a. The various embodiments may further have reservoirs for processing liquids, which may be used regardless of the format of the chip.

FIG. 2 is a schematic illustration of a patterned film 10 suitable for pneumatic (off-chip) pumping, or hydraulic, syringe-based injection. A chip formed with film 10 might also be driven suitably with a variety of electroosmotic, EWOD on-chip pumps such as Applicant's co-pending US Patent Publication 2016/051935 entitled “Peristaltic pump microfluidic separator”, or mechanical pumps, but is not suitable for centrifugal operations, given the layout. This film 10 may also be designed as for use as a stand-alone chip (requiring no other patterned film to be joined in a sandwich structure), if so a first port 22 is provided for receiving a sample, and possibly a chasing liquid (an inert dense liquid chosen for non-reaction with the sample). The first port 22 may be a standard (e.g., Luer lock) or non-standard coupling, and may be defined by a through-hole in the film 10, or a through-hole in a cover aligned with the film 10. The port 22 may be a simple hole with a smooth cylindrical surface of known resilience, for meeting a suitable tube, or may have tube attachment rims for locking or attaching to a tube. If the chip or cover has rims or protrusions, it may be manufactured by injection molding. Alternatively any or all of the ports 22,12,23,18 may be for coupling to another microfluidic network on an adjacent film having corresponding vias in alignment.

The sample liquid will first enter a long sorting channel 25 that may function by inertial confinement, or may have an offset array of microfeatures for deterministic lateral displacement, that separate particles in the sample. As microfeatures are required for the vessels, a finesse of the patterning of the substrate is already required to permit such features. The outside streams are withdrawn into ports 23 with suitable pressure at those ports, and an inside stream is ejected into a mixing chamber 26. Once a retained fraction of the sample (the inside stream) enters the mixing chamber 26, it will begin to fill the mixing chamber. Once it fills sufficiently to meet a wall opposite the entry from the sorting channel 25, the retained fraction forms a liquid plug that separates two ports 24 that are directly coupled to the mixing chamber 26. The mixing chamber 26 may overlie a heater or cooler to apply a desired temperature in accordance with a desired protocol for the chip, and one or more liquids may be injected into the mixing chamber 26 via these two ports 24. Once the retained fraction is treated in the mixing chamber 26, a pressure in one or more of the ports 22,23,24 is made higher than that of waste port 18 (ports 12 and the rest of the ports 22,23,24 being blocked or of higher pressure than waste port 18). As a result, the treated liquid is imbibed into flow-through chamber 15, via a plurality of openings 15a. A liquid front of the treated liquid passes each vessel 16 in sequence as an air plug is withdrawn from port 18 via outlet 15b. By controlling a rate of evacuation (via the difference between the low pressure of port 18 and the higher pressure at the one or more of ports 22,23,24) a dwell time of the sample within each vessel 16 can be controlled. The higher pressure may be provided by a chasing liquid, or even a gas such as a sterile gas injected under positive pressure, or by applying a negative (relative to ambient) pressure at the waste port 18. If the chasing fluid has a density higher than the treated liquid, it may replace the treated liquid within the vessels 16. Alternatively, the bulk of the sample may be withdrawn from the chamber 15, for example with the pressure difference, leaving only filled vessels of the treated liquid, in an otherwise gaseous chamber. Subsequent heating, negative pressurization, and/or gas flow through the chamber 15 can be used to evaporate or reduce the treated liquid to increase concentration, and encourage capture of any analytes for which the vessel is functionalized. Subsequent wash stages (wash introduced via the one or more of ports 22,23,24, another of these ports, and/or one or more of ports 12) can be performed to remove any residue within the vessels 16.

Subsequently a marking process is performed, which involves injection of fluids through ports 12, passage of a liquid fluid front across the vessels 16 in sequence, and exit through waste port 18. There may be several steps in this process, and a particularly invented process is described hereinbelow for use on COC substrates. At the end of the marking process, the vessels 16 can be read-out, preferably with the naked eye, and/or from a photographic record.

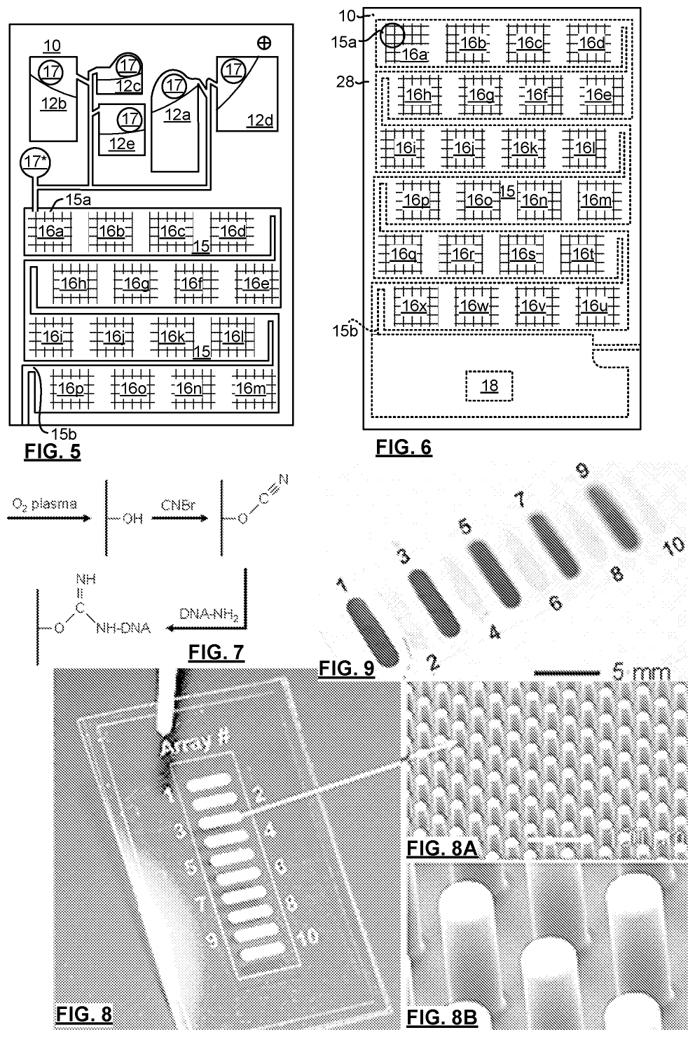

FIG. 3 is a schematic illustration of a film 10 patterned to define a centrifugal microfluidic chip designed for mounting to a centrifuge on an axis as shown. The chip's surface is partitioned into 3 areas for marking, sample prep, and readout. The chip (by virtue of a through-hole in the film 10, or a through-hole in a cover) comprises an array of 7 ports/vents: 5 controlling respective chambers 12 for performing a marking process (on the marking area), and two controlling respective chambers 14,20 (on the sample prep area). The chip may be designed for mounting to a microfluidic chip controller, according to Applicant's U.S. Pat. No. 10,702,868 “Centrifugal microfluidic chip control”, or to a 7-path rotary coupling/pneumatic slip ring. In particular, this chip, with liquids preloaded in each of these 7 chambers, is designed for complete dispensation with a suitable air plug and a controllable valve mounted to the centrifuge. This particular control strategy is particularly suited to simple processes and chips with inexpensive centrifuges and chip controllers, and can be automated for performance during a continuous centrifugation process without user intervention. For such applications, it is particularly useful to provide the chips preloaded with all liquids in an enclosed device, except for the test sample, which gets introduced via resealable covering 19, prior to mounting to a portable centrifuge for automated processing.

The sample prep area includes a chamber 20, and sample chamber 14. The chamber 20 and sample chamber 14 jointly feed a mixing chamber 26 in a particular manner that allows for highly efficient droplet mixing according to the teachings of Applicant's L. Clime, T. Veres “Centrifugal microfluidic mixing apparatus and method” CA2864641. Specifically a constriction at the entrances to the mixing chamber 26 from both chambers 14,20 results in fluid being dispensed as a discrete sequence of droplets. The droplets fall under the centrifugal force, and slide down an inclined surface. The tiny volumes of these droplets encounter one another and diffuse quickly as they have very high surface area to volume ratios, and fall into a belly of the mixing chamber 26 in a well mixed state. Once the mixture fills the belly, and primes a siphon valve, the mixture is ejected all at once into the flow-through chamber 15, filling the flow-through chamber. A fill line is shown for this chamber. As long as a total volume of liquid in chambers 14 and 20 are more than enough to fill the belly once, and not enough to fill it a second time, there will only be a single dispensation of a metered volume.

As such, rote operation of the chip defined by film 10 will initially involve opening the two right ports, to allow release of liquid in both chambers 20 and 14, while the loaded chip is under centrifugation. The two right ports may be operated by a common valve that is preferably located on a chip holder or chip controller such that the valves are never contaminated by any fluid and can be used on many chips in sequence. Release to ambient allows the fluid to drop into the constriction, and drip into the mixing chamber 26, where the droplets are mixed and accumulate in the belly until full. The fluid then empties into chamber 15, where it wets the vessels 16 in sequence, and preferably fills the volume of chamber 15. Subsequently one chamber 12 at a time is released to ambient to allow complete dispensation of the fluid contained therein, which flows directly into the chamber 15, displacing the sample and forcing the sample to exit via a drip end that conforms with a device taught in Applicant's co-pending PCT/IB/2019/059715 entitled “World-to-chip automated interface for centrifugal microfluidic platforms”. Each dispensation from chamber 12 may fill the whole chamber 15, or may produce a train of fluid segments that each treat each vessel 16 in sequence.

FIG. 4 is a variant of the film 10 of FIG. 3, adapted for a different control strategy. Instead of pressure control at valves, dispensation is controlled by respective hydrodynamic restrictions on the sample prep area, and angular tilting of the chip relative to the centrifugal field defined by an axis that is above the chip as illustrated. The angular tilting is provided by swivel mounting of the chip on an axis (shown at top right of film 10), for example in accordance with the teachings of Applicant's co-pending US 2017/0173589 entitled “Swivel mount for centrifugal microfluidic chip”. An equally automated process for going from loaded chips to answer can be provided with this variant, by providing a control over the swivel mounting according to a prescribed process. Specifically, for the designed film 10, the process will involve a zero angle for an initial period during which the sample and content of chamber 20 are mixed, the belly is filled, and the mixture is delivered in to chamber 15, and then a sequence of tilts for delivering contents of chambers 12a-e. As shown the sequence could be in alphabetical order, or d,e,a,b,c, depending on whether the chip is first tilted right or left. Alternatively any sequence with d before e, a before b, and b before c can be performed.

While the foregoing films 10 have all provided relatively small surface areas for readout, depending on a number of analytes and a desired sensitivity, it may be preferable for a larger surface area to be devoted to readout. A multistage protocol for sample preparation can be provided on a parallel layer of a centrifugal microfluidic chip without reducing a footprint of the readout area. As such the chamber 15 may occupy more than half of the chip, as shown in FIG. 5. Specifically FIG. 5 is a chip designed for operation by an articulated blade, as in the example of FIG. 4, using only 5 chambers. Sample prep is provided by a separate chip layer, and may be provided by pneumatic control, or centrifugal microfluidics. If provided by articulated (swivel mount) centrifugal actuation, efforts to avoid dispensing in the marking area during sample prep processing. With few chambers, it is relatively easy to assign dispensing tilt angles to respective processes. Once the sample prep process is completed, the liquid to be tested is supplied through a via to port 17* under centrifugation at zero tilt angle. For easier appreciation of the angle at which dispensation occurs, each of the chambers 12 has an identified fill level. It will be noted that the fill level aligns with an edge of an open port 17 such that each chamber may be designed to be overfilled, and during centrifugation, excess will leak out at the top surface. A top surface may also be patterned to avoid cross contamination if that is a risk. It will be noted that a small clockwise rotation of the swivel mount about the axis on top right will dispense chamber 12a, a larger clockwise rotation, will dispense chamber 12b, and progressively larger tilt angles in the counterclockwise direction dispense chambers 12c,d,e.

FIG. 6 is a schematic illustration of a chip consisting of a patterned film 10 covered by a cover 28 that is shown transparent to offer a ghost view of the patterned film 10. A via 15a is the only feature shown on the cover 28, although the cover could be patterned on a near side to produce any microfluidic structure required for sample preparation and/or for marking. The chamber 15 occupies a vast majority of the film 10, sharing this space only with waste reservoir 18. In other embodiments, the chip may have an exit port similar to the embodiments of FIGS. 2-5, to avoid a need for the waste reservoir 18, and thus the chamber 15 can substantially occupy the whole chip's surface area on one layer.

While the cover 28 is shown transparent, to provide the ghost view, and bearing a via serving as entry 15a into the chamber 15, it is logically preferable for an outside film of the chip to be transparent for inspection, such as reasonably transparent across a visible spectrum or at least for colors of the marking. Furthermore it is preferable for the cover, as shown, to be substantially opaque at those wavelengths, to provide contrast for the color(s). It will be appreciated that a stack of several layers may be used to produce chips according to the present invention.

While film 10 of FIG. 6 is not a complete embodiment of the instant invention, it will be appreciated that any chip including the film 10 that has two or more fluidic paths leading from respective reservoirs or supplies to the via 15a, and preferably at least three, or at least four such fluidic paths, will present a complete embodiment.

FIG. 7 is a schematic illustration of an activation scheme for a patterned film (substrate) used for a particular marking process defining a colorimetric assay, found to work well on patterned films com posed of COC. The process begins with preparation of the film for, and immobilization of amino-modified oligonucleotide probes. The surface preparation comprises the following steps:
Expose substrate (vessel regions) to oxygen plasma to release hydroxide groups (first stage of FIG. 7)
Expose freshly oxidized substrates to a 0.5 M solution of cyanogen bromide (CNBr) in acetonitrile at room temperature e.g. for a duration of 5 to 10 min
Rinse substrate with cold acetonitrile and dry with a stream of nitrogen gas (second stage of FIG. 7)
Apply respective target-bearing solutions of amino-modified oligonucleotide probes [e.g., 100 pM in deionized water] to pillar arrays: the solutions propagate across the array by capillary forces to fill the vessels
Allow DNA solution to dry out, to form spotted vessels on the film
Incubate spotted film at 95° C. for 10 min to complete binding
Rinse substrates 5 times with PBST (0.01 M phosphate-buffered saline, pH 7.2, containing 0.05% v/v Tween 20) to wash excess oligonucleotide molecules off the surface, followed by drying with a stream of nitrogen gas (third stage of FIG. 7)

At this stage the vessels are individually spotted and can be stored for several months before use. The storage can be before or after loading the chip, or even forming the chip by bonding at least a cover to the chip. The chip may advantageously be bonded to a COC substrate if the cover 28 is composed of a thermoplastic elastomer, such as taught in Applicant's U.S. Pat. Nos. 9,238,346 and 10,369,566.

Loading of the chip, according to this colorimetric marking process, involves loading into respective chambers 12:
1) Transfer 50 μL of DIG-labelled multiplex PCR products in a 1.5 mL microcentrifuge tube (use ‘lid-locks’ or screw-cap tubes to prevent the caps from popping while heating). Denature PCR products in a heating block at 100 ° C. for 10 min, followed by snap-chilling on ice water for 5 min. Pulse spin tubes in a centrifuge to bring down condensation before opening the tubes during the next step.
2) Add 150 μL of ice-cold hybridization solution (HS) containing 50% formamide to the 50 μL of denatured, labelled PCR products.
3) Saturate the pillar arrays with the HS+50% formamide+PCR product and incubate for 20 min at 45 ° C. Wash the substrates 5 times with PBST.
4) Add a freshly prepared solution of anti-DIG-POD in 0.5% PBST-B (1:2,000 v/v) to the substrate. Incubate for 10 min at room temperature. Wash the substrate 5 times with PBST.
5) Add 3,3′,5,5′-tetramethylbenzidine (TMB) membrane peroxidase substrate. Protect from light. Let the color develop for 10 min or so. Take photographs of the samples using a camera or documentation system on a UV transilluminator.

An example of a colorimetric assay performed on elongated micropillar arrays is shown in FIGS. 8,9. FIG. 8 is a photograph of a Zeonor™ COC substrate containing vessels produced by hot embossing along with scanning electron micrographs showing a close-up view of the microstructured area. The pillars are arranged in a lattice. FIG. 9 is a photograph showing good contrast and accuracy of the blue-colored spots and high specificity between spotted (odd-numbered) and non-spotted (even-numbered) regions.

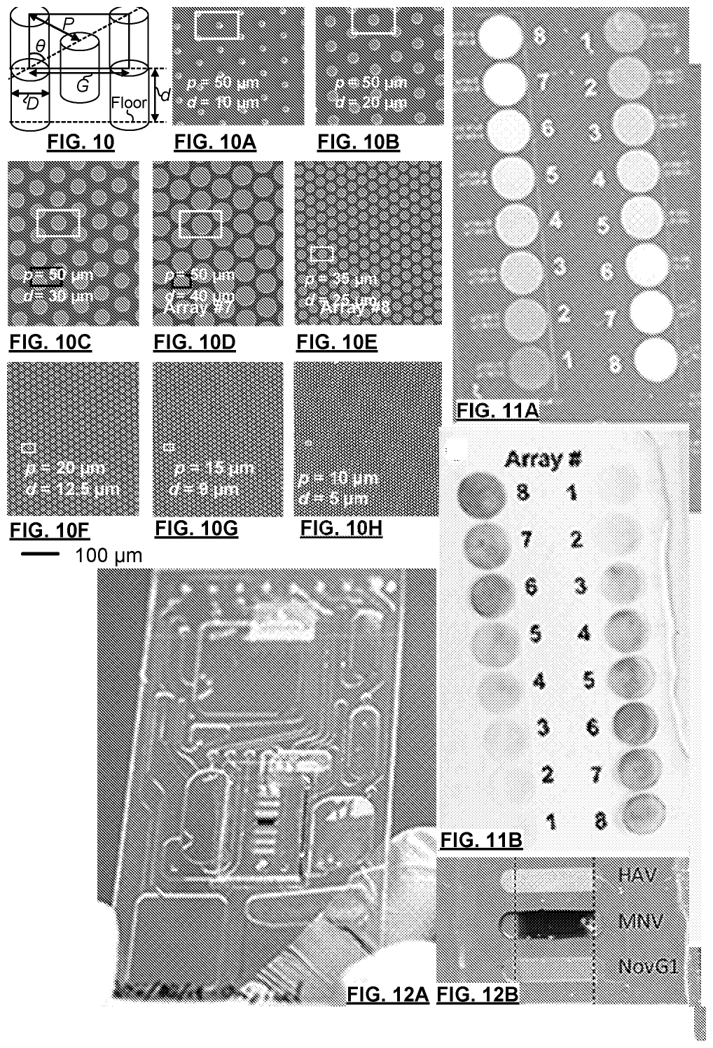

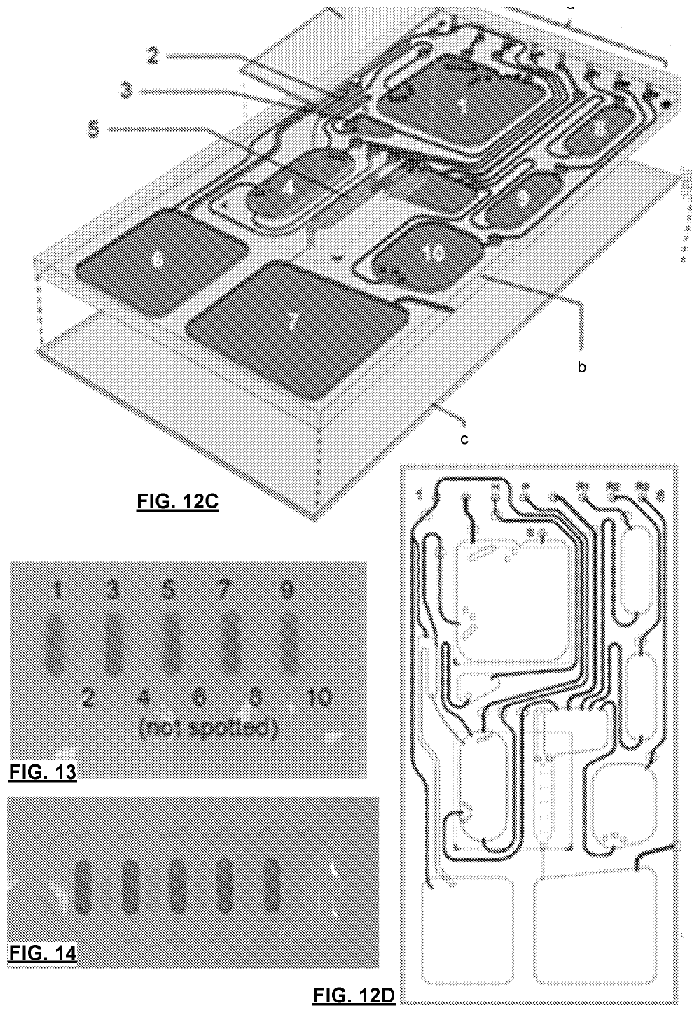

From a design point of view, micropillar arrays can generally be reduced to a particular type of unit cell, which repeats itself multiple times in both x- and y-directions. The characteristic parameters for the array are also found in the unit cell. This includes the dimensions of each pillar as well as its position with respect to neighboring pillars. FIG. 10 shows an example of a pillar array unit with hexagonal packing. Here, each pillar (which has a circular footprint) is characterized by the diameter D and the depth d. Their configuration within the unit cell is defined by the pitch P, the inter-pillar distance G, and the tilt angle θ (which is 60° in the case of a hexagonal packing). All parameters determining the unit cell can be varied either through the design (D, P, G, and θ) or through the fabrication conditions (d). In this way, the layout of an array can be chosen to achieve desired conditions for reactivity and visualization. The colorimetric signal therefore should be tunable by the array design. Preliminary results depicted in FIG. 10A-H confirm this hypothesis. The structures included in this design indicate that an increase in surface area (through variation of P and D) leads to higher signal intensities.

It is further possible to emphasize the vertical portions of the pillars (e.g., by introducing a pyramidal structure ora star-shaped cross-sectional profile, or rounded cone). Such a configuration would enable to potentially collect higher signals by emphasizing the vertical portions of each pillar.

Further, the wide area form (the geometrical confinement) of the pillar structures can be itself arranged into a shape, image, letter, number or other character to aid visualization and increase ease of user interaction. This may be particularly useful for testing by non-trained personnel, and home-based testing.

Micropillar arrays are also suitable for other, non-colorimetric detection schemes (e.g., based on fluorescence or surface plasmon resonance). Micropillar arrays induce roughness to the substrate which can lead to Mie scattering. This effect is advantageous for improving contrast and signal intensity on an otherwise transparent and colorless substrate that offers poor contrast.

Fabrication of pillar arrays in polymer materials is scalable at relatively low cost. They should therefore provide a suitable alternative to paper or other reaction matrices currently used for colorimetric assays.

Micropillar arrays also facilitate integration in a polymer-based, microfluidic chip which can be envisaged either as an insert or by embossing features simultaneously with the fluidic structures. A suitable chip design has been conceived and a first series was fabricated. Preliminary results indicate that discrimination of virus through on-chip RNA extraction and amplification is possible using a colorimetric detection assay.

FIG. 11A shows a photograph of a Zeonor substrate with micropillar arrays of different configuration produced by hot embossing. The configuration of each array number is depicted respectively in FIGS. 10A-H. FIG. 11B shows a photograph of a micropillar array substrate showing contrast provided by TMB development according to an suboptimal protocol. Arrays were all modified with O157 oligonucleotide probes. The substrate was exposed to DIG-labelled rfbO157 gene targets.

FIGS. 12A,B show a sample-to-answer chip for RNA-based virus detection. The chip has been used to perform sample lysis, multiplex PCR amplification and colorimetric identification. The array has been prepared for detecting 5 different target species simultaneously. In this example, a blue colored bar indicates the presence of MNV. The chip was operated with a centrifugal platform (Applicant's co-pending US2017/0036208 entitled Centrifugal Microfluidic Chip Control, the entire content of which is incorporated herein by reference).

Subsequent to the provisional filing, Applicant has produced further examples of the present invention. The use of polymer substrates other than Zeonor (polystyrene and polylactic acid) was demonstrated. Functionalizing different substrates may call for different processes.

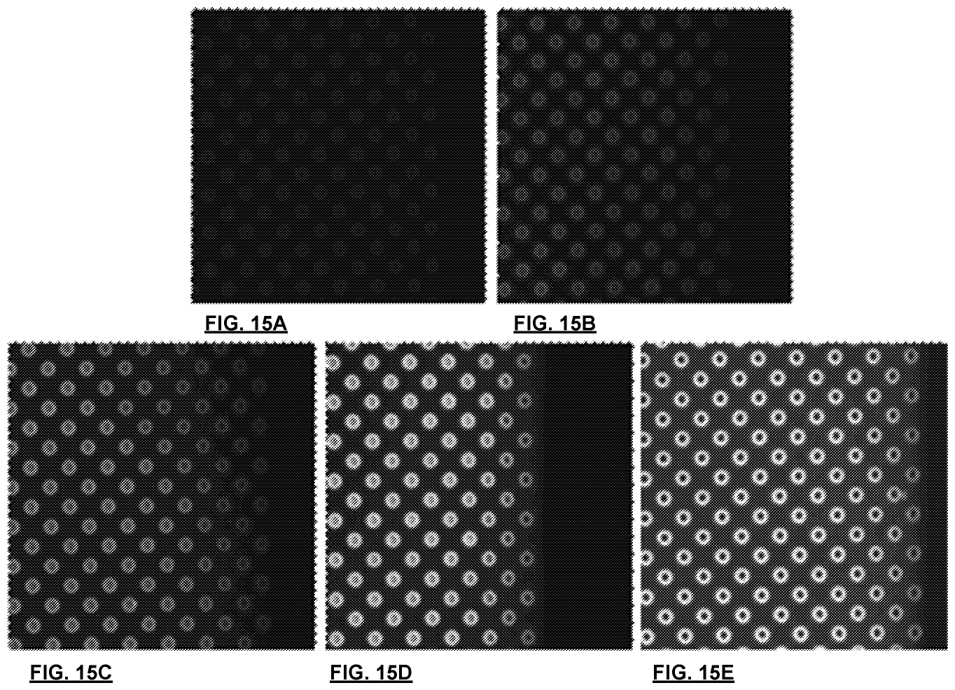

The use of activation schemes other than oxygen plasma and cyanogen bromide treatment has been demonstrated. Polylactic acid micropillars were modified using combined UV/ozone treatment followed by reaction with amino propyl triethoxysilane and glutaraldehyde. Applicant has successfully employed UV/ozone treatment on polystyrene as an alternative to oxygen plasma treatment. Polystyrene micropillars were modified with oxygen plasma treatment, followed by reaction with amino propyl triethoxysilane and glutaraldehyde. The micropillars were then spotted with biotinylated antibody (Goat pAB against Human Albumin) which was then revealed through HRP-conjugated streptavidin and TMB conversion. FIGS. 13,14 respectively illustrate the contrast produced on the polylactic acid pillars, and polystyrene micropillars, respectively. Unfunctionalized vessels were interleaved between the functionalized vessels to show contrast provided by the target reporting.

Applicant has further demonstrated fluorescence-based detection as opposed to colorimetric detection. While colorimetric detection involves production of a dye that can typically multiply a strength of the signal offered by a reporting target, fluorescent labelling allows only the emissions of the target particles to report. The density and arrangement of target particles around micropillars provides a surprisingly strong observable signal. Fluorescence micrographs of a micropillar array (Zeonor) modified with a biotinylated antibody (Goat pAB against Human Albumin) at different concentrations and used in a fluorescence binding assay with Cy3-labelled streptavidin. The Zeonor surface was activated using oxygen plasma treatment, followed by reaction with amino propyl triethoxysilane and glutaraldehyde. Fluorescence intensity was found to grow exponentially as a function of probe concentration. FIGS. 15A,B,C,D,E form a panel showing fluorescent response of vessels exposed to 1, 5, 10, 50, and 100 μg/ml concentrations of albumin.

In greater detail, the examples were produced as follows:
1) Microstructured Zeonor, polystyrene or polylactic acid substrates were exposed to oxygen plasma for 2 min at a pressure of 50 mTorr, a power of 100 W, and a gas flow of 20 sccm. Some polystyrene substrates were alternativelyexposed to UV/ozone for 10 min.
2) Freshly oxidized substrates were incubated with a 2% aqueous solution of amino propyl triethoxysilane for 1 h at RT. Samples were rinsed with DI water and dried with a stream of nitrogen gas.
3) Samples were incubated with a 2% solution of glutaraldehyde in PBS for 2 h at RT. Samples were rinsed with DI water and dried with a stream of nitrogen gas.
4) Capture AB (200 pg/mL) was spotted on alternating interface pinning reaction vessels using slotted pins (500 nL capacity). The samples were stored in a Petri dish containing wet strips to maintain humidity. The Petri dish was sealed to prevent evaporation and stored. Incubation times were from 1 h to 12 h.
5) Substrates were immersed in PBST+Casein blocking solution 1:1 (v/v) for 1 min and then rinsed with DI water and dried. No additional blocking step seems to be necessary. If rinsing is done without blocking agent, traces of protein from the spotted region to the outside areas are apparent on the sample since the glutaraldehyde is still reactive at this stage.
6) Antigen solution is added for capture. Streptavidin-HRP as well as Streptavidin-Cy3 have been used in conjunction with a biotin label for colorimetric of fluorescence detection. Incubation for forming the biotin/streptavidin complex lasted for 5 min (about 200 μL per sample).
7) Substrates are rinsed with PBST+Casein blocking solution 1:1 (v/v), DI water and dried with a stream of nitrogen gas.
8) Samples with HPR-modified streptavidin were incubated with TMB membrane peroxidase substrate for up to 1 h. Samples treated with Cy3-labelled streptavidin were inspected using fluorescence microscopy.

Applicant has thus described a variety of embodiments of the present invention, and further demonstrated the ability to selectively report targets on interface-pinning reaction vessels. Other advantages that are inherent to the structure are obvious to one skilled in the art. The embodiments are described herein illustratively and are not meant to limit the scope of the invention as claimed. Variations of the foregoing embodiments will be evident to a person of ordinary skill and are intended by the inventor to be encompassed by the following claims.

1. A kit for forming a microfluidic chip, the kit comprising:
a substrate having a surface with topographical relief bearing at least 4 relief patterned regions, each defining a respective interface-pinning reaction vessel covering a footprint area of 0.5 to 15 cm²; and
a part with a covering surface dimensioned for sealing against the substrate to enclose a single flow-through chamber that includes the vessels.

2. The kit according to claim 1 wherein each region extends 0.1 to 50 mm in both planar directions.

3.-4. (canceled)

5. The kit according to claim 14 wherein each region has a surface area that is 2-50 times its footprint area.

6. The kit according to claim 1 wherein each region is separated from each neighbouring region by segments of the surface that have a ratio of surface area to footprint that is no more than 1.1 and each segment separates the neighbouring regions by a distance that is greater than at least one of: 0.1 mm; or 5% of a mean of the extents of the neighbouring regions in the planar directions.

7. (canceled)

8. The kit according to claim 1 wherein the chamber has at least one ingress from a microfluidic network of a chip that includes the substrate and the part, the microfluidic network comprising at least two microfluidic channels coupling two different reservoirs with the ingress.

9. The kit according to claim 8 wherein the microfluidic network comprises two subnetworks: a marking network equipped for performing a marking process within the chamber; and a prep network equipped for treating a test sample.

10. The kit according to claim 8 wherein the part is a first film, and the covering surface is a side of the first film.

11. The kit according to claim 10 wherein the side of the first film, or the substrate surface, is relief patterned to define one or more of: one side of the chamber, the relief-patterned regions, one side of the ingress, the whole ingress defined as a throughbore of the first film, and at least part of the microfluidic network.

12. The kit according to claim 10 wherein the relief pattern defines at least one microfluidic blister for retaining a liquid.

13. The kit according to claim 10 wherein the substrate is a second film; the kit further comprises a third film; and at least one of the first, second or third films, has at least one through-bore via for coupling two microfluidic networks when stacked.

14. The kit according to claim 1 wherein at least one of the substrate and the part is transparent to an inspection wavelength; a chip produced by sealing the surface and the covering surface permits inspection of the vessels through the transparent material; and the transparent material is sealed to a material that is reflective or opaque to the inspection wavelength, to improve imaging of the vessels.

15. (canceled)

16. The kit according to claim 1 further comprising supplies of at least 3 probes.

17. The kit according to claim 16 wherein the supplies are provided by functionalizing each of the vessels with a respective one and only one of the at least 3 probes.

18. The kit according to claim 17 wherein the substrate is composed of a cyclic olefin copolymer, and the functionalization is consistent with formation by oxygen plasma surface activation, reaction with cyanogen bromide, and binding of the probe.

19. The kit according to claim 16 wherein the supply is provided, carried by a liquid in a fluid-tight container, the liquid having a contact angle and viscosity allowing for spontaneous spreading of the liquid across the region, and a volume sufficient to cover the region, but insufficient volume to overcome interface pinning, whereby the liquid, if it meets any part of the region, is self-limited to substantially covering that region.

20. The kit according to claim 1 further comprising at least one marking liquid the marking liquid comprising one or more of: a developer; a conjugated detection antibody with a target-specific binding moiety; a wash buffer; a hybridization solution; formaldehyde; and a PCR product contained within a microfluidic chamber of a chip formed with at least the substrate and the cover.

21. (canceled)

22. The kit according to claim 20 wherein the substrate is a cyclic olefin copolymer, and the developer is 3,3′,5,5′-tetramethylbenzidine.

23. The kit according to any one of claims 1 to 22 assembled to form a chip.

24. A method for assaying on a microfluidic chip, the method comprising:
providing a microfluidic chip, the chip having at least one flow-through chamber having, on a single surface thereof composed of a cyclic olefin copolymer, a topographical relief bearing at least one relief patterned region:
defining a respective interface-pinning reaction vessel; and
functionalized with a respective probe;
supplying a test sample into the flow-through chamber, so that the test sample flows over each of the regions;
supplying rinse buffer to wash unbound analyte off the surface;
supplying a detection antibody conjugated with an enzyme;
supplying rinse buffer to wash excess detection antibody off the surface;
supplying a developer to all vessels by flowing a developer agent through the chamber.

25. The method of claim 24:
further comprising functionalizing the respective regions by dispensing a droplet anywhere within the region, allowing the droplet to spread across the region, evaporating the solvent, heating the region to above 60° C. for 1-10 min, rinsing with buffer with a surfactant, and drying, prior to forming the chip by enclosing a relief-patterned substrate;
wherein rinsing with buffer comprises:
dispensing a droplet of the buffer with surfactant into each region respectively, allowing the buffer to dissolve or suspend any unbound probe or reaction product, and wicking the buffer out of each of the regions without mixing the respective droplets; or
flooding the regions with the buffer and surfactant, allowing the dissolution or suspension of any unbound probe or reaction product, and extracting the buffer from the regions;
wherein the developer agent produces a dye that is insoluble in a cleaning solution, but the developer agent is soluble in the cleaning solution, and the method further comprises flowing the cleaning solution through the chamber after supplying the developer; or
wherein the developer comprises TMB; the rinse buffer is PBST; a hybridization solution containing formaldehyde; and the conjugated detection antibody has a target-specific antibody moiety and a conjugated HRP enzyme.

26.-28 (canceled)
